(19) United States
(12) Patent Application Publication
(10) Pub. No.: US 2025/0339543 A1
(43) Pub. Date: Nov. 6, 2025

(54) POLYMER-BASED CONJUGATE, COMPRISING POLYPROPYLENE OXIDE, FOR IMPROVING THERAPEUTIC EFFECTS OF ANTIBODIES FOR TREATING AUTOIMMUNE DISEASES

(71) Applicant: THE CATHOLIC UNIVERSITY OF KOREA INDUSTRY-ACADEMIC COOPERATION FOUNDATION, Seoul (KR)

(72) Inventors: Kun Na, Gyeonggi-do (KR); Jae Won Roh, Gyeonggi-do (KR)

(21) Appl. No.: 18/864,441

(22) PCT Filed: May 11, 2023
(86) PCT No.: PCT/KR2023/006411

§ 371 Date: Nov. 8, 2024

(30) Foreign Application Priority Data
May 12, 2022 (KR) 10-2022-0058579

(51) Int. Cl. A61K 47/60; A61K 39/00; A61P 37/06; C07K 16/24

(52) U.S. Cl. CPC A61K 47/60; A61P 37/06; C07K 16/241; A61K 2039/505; C07K 2317/76

(57) ABSTRACT

The present disclosure relates to: a block polymer conjugate comprising an antibody-linker-polypropylene oxide, in which block polymer comprising polypropylene oxide is conjugated to an antibody through a linker; and pharmaceutical composition for treating an autoimmune disease, comprising the conjugate. The block polymer conjugate comprising an antibody-linker-polypropylene oxide, according to the present disclosure, exhibits the effects of maintaining specific reactions of conventional antibodies, simultaneously, increasing the stability from proteolytic enzymes and improving in vivo half-life. When the conjugate according to the present disclosure is administered to a disease site, effects can be exhibited for longer than those of a conventional antibody and biostability and anti-inflammatory effects greater than those of a conventional antibody are exhibited.

### TECHNICAL FIELD

The present disclosure relates to a conjugate comprising an antibody-linker-polypropylene oxide-containing block polymer and a composition comprising the same for preventing or treating an autoimmune disease.

### BACKGROUND ART

Autoimmune diseases are treated with a variety of therapies, depending on the stage of the disease, and the patient's response or tolerance to the drug is a key factor in choosing a treatment. Traditionally, non-steroidal anti-inflammatory drugs that inhibit cyclooxygenase (COX) proteins or gluticorticoid drugs that inhibit receptors for cytokines involved in inflammatory signaling have been used. Currently, methotrexate, the most commonly used drug, or its combination with other drugs, is the main recommendation.

However, when the patient is resistant to these drugs or the disease has progressed to a severe level, biological agents are prescribed. The biological agents include antibody-based therapeutic agents that inhibit inflammatory cytokines or their corresponding proteins that are overexpressed at the lesion site.

In particular, antibodies that specifically act on TNF-α, which are involved in the NF-κB signaling pathway, which plays a central role in the inflammatory response, are mainly used. TNF-α is secreted in immune cells, such as macrophages and T cells, which binds to TNF-α receptors expressed on most cells and activates the intracellular NF-κB signaling pathway. It then exhibits several inflammatory actions, including the secretion of inflammatory cytokines, activation of inflammatory regulatory complexes, and proliferation and differentiation of immune cells, etc. For this reason, NF-κB is considered one of the hallmarks of inflammatory diseases.

For this reason, TNF-α-specific antibody therapeutic agents are continuously being studied, and many have already been approved by the FDA. Currently approved TNF-α-specific antibodies include Adalimumab, Infliximab, Golimumab, Etanercept, and the like. Despite multiple conditions, these drugs remain among the top-selling blockbuster drugs in the world today, and biosimilar approvals and trials for these drugs are ongoing.

However, the antibody therapeutic agents raise concerns about the potential for autoimmunity leading to the production of anti-drug antibodies. The anti-drug antibodies not only reduce the effectiveness of the drug, but also induce an antibody-based immune response, suggesting the potential for another inflammatory response to be induced. Therefore, a major challenge is to improve the therapeutic effect by reducing autoimmunity caused by the nature of the antibody therapeutic agents themselves.

Therefore, the present inventor has developed an antibody conjugate capable of interacting specifically with TNF-α by conjugating a TNF-α-specific antibody to polyethylene oxide (PEO) and polypropylene oxide (PPO) polymer via a linker. The PEO and PPO copolymer is FDA-approved as pharmaceutical adjuvants due to high biocompatibility, low toxicity, and rheological properties suitable for use in the body. The PEO and PPO copolymer is known to reduce tissue injury caused by inflammation at the lesion site and have anti-inflammatory effects on their own. In this regard, the present inventors found that conjugation of PEO and PPO copolymers to antibodies increased their in vivo half-life compared to conventional antibodies, preventing the antibodies from being metabolized and maintaining their anti-inflammatory effects, and completed the present disclosure.

### DISCLOSURE

### Technical Problem

An object of the present disclosure is to provide a conjugate comprising: (a) an antibody; (b) a linker linked by a covalent bond to the antibody; and (c) polyethylene oxide (PEO) and polypropylene oxide (PPO) polymer linked by a covalent bond to the linker.

Another object of the present disclosure is to provide a method for producing a conjugate, the conjugate comprising: (a) an antibody; (b) a linker linked by a covalent bond to the antibody; and (c) polyethylene oxide (PEO) and polypropylene oxide (PPO) polymer linked by a covalent bond to the linker.

Still another object of the present disclosure is to provide a pharmaceutical composition comprising a conjugate for preventing or treating an autoimmune disease, the conjugate comprising: (a) an antibody; (b) a linker linked by a covalent bond to the antibody; and (c) polyethylene oxide (PEO) and polypropylene oxide (PPO) polymer linked by a covalent bond to the linker.

### Technical Solution

In the following specification, description of overlapping content will be omitted to prevent any potential confusion arising from redundancy. In other words, the content of the disclosure is not limited to the following content; rather, it should be construed in accordance with the comprehensive content of the disclosure.

Further, terms used herein are merely used for illustration purposes, which should not be construed as limiting the present disclosure. Singular expressions include plural expressions unless the context clearly indicates otherwise. In the present specification, terms such as “comprise” or “have” are intended to designate the presence of features, numbers, steps, operations, components, parts, or combinations thereof as described in the specification without precluding the possibility of the presence or addition of one or more other features, numbers, steps, operations, components, parts, or combinations thereof.

Further, unless defined otherwise, all terms used herein, including technical or scientific terms, have the same meaning as commonly understood by one of ordinary skill in the art to which the embodiments belong. Terms such as those defined in commonly used dictionaries should be interpreted as having a meaning consistent with the meaning in the context of the related art, and unless explicitly defined in the present application, it is not to be construed in an idealized or overly formal sense.

In one general aspect, there is provided a conjugate comprising: (a) an antibody; (b) a linker linked by a covalent bond to the antibody; and (c) polyethylene oxide (PEO) and polypropylene oxide (PPO) polymer linked by a covalent bond to the linker.

The term “antibody” in the present disclosure may comprise fragments of antibody molecules as well as complete forms having two full-length light chains and two full-length heavy chains. In addition, antibodies include polyclonal antibodies, monoclonal antibodies, chimeric or chimeric antibodies, humanized antibodies, primitive antibodies, deimmunized antibodies, and fully human antibodies. The antibodies can be produced in or derived from any of a variety of species, e.g., mammals, such as humans, non-human primates (e.g., orangutans, baboons, or chimpanzees), horses, cows, pigs, sheep, goats, dogs, cats, rabbits, guinea pigs, gerbils, hamsters, rats, and mice. The antibody may be a purified or recombinant antibody.

In an embodiment of the present disclosure, the antibody may be a TNF-α specific antibody. In other words, the antibody may be an anti-TNF-α antibody.

TNF-α is a cytokine produced by multiple cell types, including monocytes and macrophages, and has been suggested to play a role in the pathophysiology of a variety of other human diseases and disorders, including shock, sepsis, infection, autoimmune diseases, rheumatoid arthritis (RA), Crohn's disease, graft rejection, and graft-versus-host disease. To inhibit this TNF-α activity, TNF-α-specific antibodies that bind to TNF-α and induce neutralization are being used as therapeutic agents. FDA-approved TNF-α-specific antibodies include, for example, Adalimumab, Infliximab, Golimumab, Etanercept, Certolizumab pegol, and the like. These antibodies specifically neutralize TNF-α at the site of inflammation, thereby inhibiting the progressio2n of inflammation. Since autoimmune diseases usually have chronic symptoms, a polymer containing polypropylene oxide is capable of improving therapeutic effects due to increased half-life and stability in the body.

In an embodiment of the present disclosure, the TNF-α specific antibody may be any one or more selected from the group consisting of Adalimumab, Infliximab, and Golimumab, all in monoclonal IgG form.

The “linker” of the present disclosure refers to a substance that links an antibody to polyethylene oxide (PEO) and polypropylene oxide (PPO) polymer. As used herein, the linker may be a linker capable of being degraded by an external stimulus, such as a peptide group including dipeptides, a carbohydrate group including disaccharides, a phosphate group including phosphates and pyrophosphates, a sulfate group, etc., and preferably, any one selected from the group consisting of maleimide-thiol, thiol, maleimide, succinic anhydride, N-hydroxysuccinimide ester, carboxyl-amine, hydrazone, and disulfide bonds.

More preferably, in an embodiment of the present disclosure, the linker may be a maleimide-thiol, which is a typical click chemistry reaction, has the advantage of being able to react in a buffer. The thioether bond formed in the maleimide-thiol is highly stable and is unlikely to degrade, thereby maintaining the effectiveness of polypropylene oxide-containing polymers for a long time to increase half-life and stability in the body.

As used herein, the term “PEO (polyethylene oxide) and PPO (polypropylene oxide) polymer” refers to a polymer that alternately contains polypropylene oxide polymer and polyethylene oxide polymer. More specifically, the “block copolymer containing PEO and PPO (hereinafter, referred to as PEO and PPO-containing block copolymer)” refers to a copolymer alternately comprising polyethylene oxide blocks and polypropylene oxide blocks.

Preferably, the PEO (polyethylene oxide) and PPO (polypropylene oxide) polymer may be PEO-PPO-PEO polymer, which is a terpolymer alternately containing polyethylene oxide polymers.

As used herein, the polyethylene oxide (PEO) and polypropylene oxide (PPO) polymer may have a weight average molecular weight of 1 kDa to 20 kDa, preferably 3 kDa to 17 kDa, more preferably 5 kDa to 15 kDa, and even more preferably 7 kDa to 13 kDa. Here, 1 kDa is 1000 g/mol.

In addition, specifically, the polyethylene oxide (PEO) and polypropylene oxide (PPO) polymer may include polypropylene oxide (PPO) having a weight average molecular weight of 1 kDa to 5 kDa, preferably 1.3 kDa to 4.5 kDa, and more preferably 1.5 kDa to 4 kDa.

Preferably, examples of commercially available PEO (polyethylene oxide) and PPO (polypropylene oxide) polymers may be any one selected from the group consisting of Poloxamer 68, Poloxamer 124, Poloxamer 127, Poloxamer 184, Poloxamer 185, Poloxamer 188 (Pluronic F-68), Poloxamer 237, Poloxamer 338, and Poloxamer 407 (Pluronic F-127), but are not limited thereto. More preferably, the PEO-PPO-PEO polymer may be Poloxamer 188 (Pluronic F-68) or Poloxamer 407 (Pluronic F-127).

In the present disclosure, the antibody, linker, and polyethylene oxide (PEO) and polypropylene oxide (PPO) polymer may each be connected by a covalent bond. Here, the covalent bond may be at least any one selected from the group consisting of a thioether bond, an amide bond, a carbonyl bond, an ester bond, a thioester bond, a sulfonamide bond, and a urethane bond.

According to an embodiment of the present disclosure, specifically, an antibody-maleimide linked by an amide bond may be prepared by reacting sulfo-SMCC (sulfo-succinimidyl 4-(N-maleimidomethyl)cyclohexane-1-carboxylate) to the amine group end of the antibody, and a polypropylene oxide-containing polymer-thiol may be prepared by thiolizing an amine in a substance obtained by amidation of hydroxyl groups present at both ends of a polypropylene oxide-containing polymer. Then, these two substances may be linked by a thiol bond by inducing a click chemical reaction between the maleimide and the thiol.

According to an embodiment of the present disclosure, the conjugate according to the present disclosure may have improved half-life and stability in the body compared to existing antibodies.

According to another embodiment of the present disclosure, the conjugate according to the present disclosure may have improved regenerative effect on damaged tissue compared to existing antibodies.

In another general aspect, there is provided a method for producing a conjugate comprising: (a) preparing a first conjugate in which a PEO (polyethylene oxide) and PPO (polypropylene oxide) polymer and a linker are linked by a covalent bond; and (b) preparing a second conjugate in which the linker of the first conjugate and an antibody are linked by a covalent bond.

In still another general aspect, there is provided a pharmaceutical composition comprising a conjugate for preventing or treating an autoimmune disease, the conjugate comprising: (a) an antibody; (b) a linker linked by a covalent bond to the antibody; and (c) polyethylene oxide (PEO) and polypropylene oxide (PPO) polymer linked by a covalent bond to the linker.

The term “autoimmune disease” is a general term for a disease that occurs when the immune system attacks the body's normal tissues, organs, or other body components due to an immune system disorder of unknown origin, which is a systemic disease that can affect almost any part of the body, including the nervous system, gastrointestinal system, endocrine system, skin, skeletal system, and vascular tissue.

The autoimmune disease may be, for example, at least any one selected from the group consisting of atopic dermatitis, alopecia areata, allergy, asthma, conjunctivitis, periodontitis, rhinitis, otitis media, pharyngitis, tonsillitis, pneumonia, gastric ulcer, gastritis, Crohn's disease, psoriasis, ulcerative colitis, Behcet's enteritis, hidradenitis suppurativa, uveitis, hemorrhoids, gout, ankylosing spondylitis, rheumatic fever, lupus, fibromyalgia, psoriatic arthritis, axial spondyloarthritis, osteoarthritis, rheumatoid arthritis, periarthritis, tendonitis, tenosynovitis, peritendonitis, myositis, hepatitis, cystitis, nephritis, Sjogren's syndrome, and multiple sclerosis.

As used herein, “prevention” means any act of inhibiting or delaying an autoimmune disease by administering the composition of the present disclosure to a subject.

As used herein, “treatment” means any act of ameliorating the symptoms of an autoimmune disease or benefiting a subject by administering the composition of the present disclosure to the subject.

For the preparation of pharmaceutical compositions, the types of carrier capable of being used in the present disclosure are not particularly limited, and any carrier commonly used in the art can be used. Non-limiting examples of the carriers may include saline, sterile water, Ringer's solution, buffered saline, albumin injection solution, lactose, dextrose, sucrose, sorbitol, mannitol, xylitol, erythritol, maltitol, maltodextrin, glycerol, ethanol, and the like. These can be used alone or in combination of two or more.

The compositions of the present disclosure may also be used with other pharmaceutically acceptable additives, such as excipients, diluents, antioxidants, buffers or bacteriostatic agents, if desired, and may additionally be used with fillers, extenders, wetting agents, disintegrants, dispersants, surfactants, binders or lubricants.

The compositions of the present disclosure may be formulated and used in a variety of suitable dosage forms for oral or parenteral administration, but more preferably in a dosage form for parenteral administration, and even more preferably as an injectable or infusion.

To formulate the compositions of the present disclosure for parenteral administration, sterile aqueous solutions, non-aqueous solvents, suspensions, emulsions, lyophilized preparations, external preparation, and the like, may be used. The non-aqueous solvents and suspensions may include propylene glycol, polyethylene glycol, vegetable oils such as olive oil, and injectable esters such as ethylolates, and the like. Furthermore, when formulated as an injectable or infusion, the composition of the present disclosure may be mixed in water with a stabilizer or buffer to form a solution or suspension and formulated for unit dosing in an ampoule or vial.

In addition, the composition may be transplanted and administered using an administration method commonly used in the art, and preferably, may be directly engrafted or transplanted into a disease site of the patient in need of treatment, but the present disclosure is not limited thereto. For example, the composition of the present disclosure may be administered rectally, subcutaneously, intramuscularly, intraperitoneally, intravenously, intraarterially, intrathecally, intramedullary, etc., and preferably intravenously. In addition, the administration may be performed by both non-surgical administration using a catheter and surgical administration such as injection or transplantation after incision in the disease site.

However, it should be understood that an actual dosage of an active component should be determined in light of various relevant factors such as a disease to be treated, severity of the disease, a route of administration, a weight, age and sex of the patient, and the like, and thus the dosage does not limit the scope of the present disclosure in any way.

The effective amount, i.e., the effective dosage, of the composition of the present disclosure may vary depending on by the method of formulation of the composition, the mode of administration, the time of administration, and/or the route of administration, and may be varied by the kind and degree of response desired to be achieved by administration of the composition, the kind of individual to be administered, age, weight, general state of health, symptoms or degree of disease, sex, diet, excretion, ingredients of other compositions together used simultaneously or at different times with the individual, and similar factors well known in the pharmaceutical field, and a person skilled in the art will be able to readily determine the dosage such that the desired effect is fully achieved.

The present disclosure also includes the following embodiments:
a conjugate for use as a pharmaceutical agent, comprising: (a) an antibody; (b) a linker linked by a covalent bond to the antibody; and (c) polyethylene oxide (PEO) and polypropylene oxide (PPO) polymer linked by a covalent bond to the linker,
a pharmaceutical composition comprising a conjugate for use in the prevention or treatment of an autoimmune disease, the conjugate comprising: (a) an antibody; (b) a linker linked by a covalent bond to the antibody; and (c) polyethylene oxide (PEO) and polypropylene oxide (PPO) polymer linked by a covalent bond to the linker,
use of a conjugate for the manufacture of medicament for the treatment of an autoimmune disease, the conjugate comprising: (a) an antibody; (b) a linker linked by a covalent bond to the antibody; and (c) polyethylene oxide (PEO) and polypropylene oxide (PPO) polymer linked by a covalent bond to the linker,
a method for treating an autoimmune disease, comprising: administering therapeutically effective amount of a conjugate to a subject in need thereof, the conjugate comprising: (a) an antibody; (b) a linker linked by a covalent bond to the antibody; and (c) polyethylene oxide (PEO) and polypropylene oxide (PPO) polymer linked by a covalent bond to the linker, and
a pharmaceutical composition comprising: a conjugate; and a pharmaceutically acceptable carrier, the conjugate comprising: (a) an antibody; (b) a linker linked by a covalent bond to the antibody; and (c) polyethylene oxide (PEO) and polypropylene oxide (PPO) polymer linked by a covalent bond to the linker.

Matters described in the composition, use, and treatment method of the present disclosure are equally applied unless they contradict each other.

### Advantageous Effects

The block polymer conjugate comprising an antibody-linker-polypropylene oxide, according to the present disclosure, exhibits the effects of maintaining specific reactions of conventional antibodies, simultaneously, increasing the stability from proteolytic enzymes and improving in vivo half-life. When the conjugate according to the present disclosure is administered to a disease site, effects can be exhibited for longer than those of a conventional antibody and biostability and anti-inflammatory effects greater than those of a conventional antibody are exhibited.

### DESCRIPTION OF DRAWINGS

FIG. 1 shows ¹H-NMR spectra of (a) amine-polypropylene oxide 1.7 k-containing block polymer, and (b) amine-polypropylene oxide 3.8 k-containing block polymer according to an embodiment of the present disclosure.

FIG. 2 shows MALDI-TOF/MS spectra of (a) adalimumab-maleimide-thiol-polypropylene oxide 1.7 k-containing block polymer, (b) infliximab-maleimide-thiol-polypropylene oxide 1.7 k-containing block polymer, and (c) golimumab-maleimide-thiol-polypropylene oxide 1.7 k-containing block polymer according to an embodiment of the present disclosure.

FIG. 3 shows SDS-PAGE results of (a) adalimumab-maleimide-thiol-polypropylene oxide 1.7 k-containing block polymer, (b) infliximab-maleimide-thiol-polypropylene oxide 1.7 k-containing block polymer, and (c) golimumab-maleimide-thiol-polypropylene oxide 1.7 k-containing block polymer according to an embodiment of the present disclosure.

FIG. 4 shows HIC-HPLC spectrum of the adalimumab-maleimide-thiol-polypropylene oxide 1.7 k-containing block polymer according to an embodiment of the present disclosure.

FIG. 5 shows the antibody secondary structure using circular dichroism spectrometry, of (a) adalimumab-maleimide-thiol-polypropylene oxide 1.7 k-containing block polymer, (b) infliximab-maleimide-thiol-polypropylene oxide 1.7 k-containing block polymer, and (c) golimumab-maleimide-thiol-polypropylene oxide 1.7 k-containing block polymer according to an embodiment of the present disclosure.

FIG. 6 shows the stability under biological conditions of (a) adalimumab and (b) adalimumab-maleimide-thiol-polypropylene oxide 1.7 k-containing block polymer according to an embodiment of the present disclosure.

FIG. 7 shows the enzymatic protein degradation rates of (a) adalimumab and (b) adalimumab-maleimide-thiol-polypropylene oxide 1.7 k-containing block polymer according to an embodiment of the present disclosure, and (c) enzymatic protein degradation stability thereof.

FIG. 8 shows the toxic effects on normal cells of (a) adalimumab-maleimide-thiol-polypropylene oxide 1.7 k-containing block polymer, (b) infliximab-maleimide-thiol-polypropylene oxide 1.7 k-containing block polymer, and (c) golimumab-maleimide-thiol-polypropylene oxide 1.7 k-containing block polymer according to an embodiment of the present disclosure.

FIG. 9 shows flow cytometry analysis of (a) a negative control group, (b) a positive control group treated with both TNF-α and CHX, (c, e, g) a positive control group treated with the conventional antibody, and (d, f, h) a positive control group treated with an antibody-linker-polypropylene oxide-containing block polymer conjugate according to an embodiment of the present disclosure; and (i) inhibition of apoptosis through tumor necrosis factor-alpha neutralization.

FIG. 10 shows the therapeutic effect of the adalimumab-maleimide-thiol-polypropylene oxide 1.7 k-containing block polymer according to an embodiment of the present disclosure on a rheumatoid arthritis mouse model as measured by (a) a change in body weight, (b) a change in arthritis levels, and (c) a change in ankle thickness.

FIG. 11 shows the therapeutic effect of the adalimumab-maleimide-thiol-polypropylene oxide 1.7 k-containing block polymer according to an embodiment of the present disclosure on a rheumatoid arthritis mouse model as measured by (a) an X-ray tomography image and (b) a bone surface area ratio (BS/BV) analysis.

FIG. 12 shows the therapeutic effect of the adalimumab-maleimide-thiol-polypropylene oxide 1.7 k-containing block polymer according to an embodiment of the present disclosure on a rheumatoid arthritis mouse model as measured by (a) H&E staining and inflammation level, and (b) Safranin O staining and cartilage injury level.

FIG. 13 shows a change in the expression level of inflammatory cytokine by the adalimumab-maleimide-thiol-polypropylene oxide 1.7 k-containing block polymer according to an embodiment of the present disclosure.

### BEST MODE

Hereinafter, the present disclosure will be described in more detail through Examples. These Examples are provided solely for the purpose of illustrating the present disclosure, and it will be apparent to those skilled in the art that the scope of the present disclosure should not be construed as limited by these Examples.

### <EXAMPLE> PRODUCTION OF ANTIBODY-LINKER-POLYPROPYLENE OXIDE-CONTAINING BLOCK POLYMER CONJUGATE

### Example 1-1: Production and Purification of adalimumab-aleimide-thiol-polypropylene Oxide 1.7 k-Containing Block Polymer Conjugate

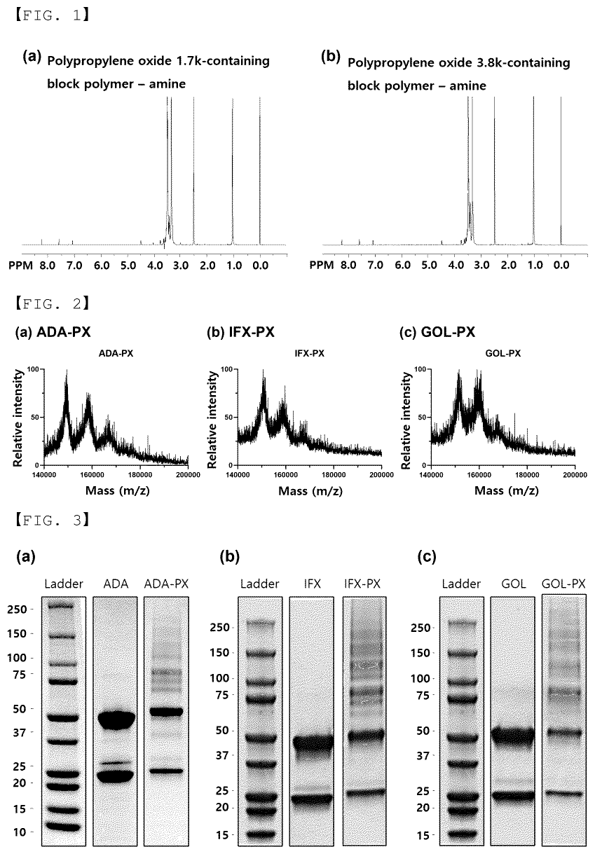

In 5 mL of dichloromethane (DCM), 1 g of polypropylene oxide 1.7 k-containing block polymer (Poloxamer 188), 480 mg of 4-nitrophenyl chloroformate (4-NPC), and 2.9 mg of 4-dimethylaminopyridine (DMAP) were dissolved and stirred at 4° C. for 30 minutes, and then stirred again at room temperature for 12 hours to react. After precipitating 5 mL of the reaction solution in 40 mL of diethylether (DE), the reaction product was subjected to centrifugation (3500 rpm, 5 minutes) to remove the supernatant and obtain the precipitate. The resulting product was centrifuged three times and then dried under reduced pressure to remove residual reagents and residual DE. The dried compound (500 mg) was dissolved in 5 ml of DCM, 75.8 μL of ethylenediamine (EDA) was then added, and the reaction mixture was stirred for about 12 hours to react. The reaction solution was dialyzed using a dialysis membrane (MWCO: 3,500 Da) for one day to obtain an amine-polypropylene oxide 1.7 k-containing block polymer, freeze-dried, and stored in powder form. The results were confirmed through nuclear magnetic resonance spectrum (¹H-NMR) as shown in FIG. 1(a).

After that, 3 mg of amine-polypropylene oxide 1.7 k-containing block polymer was dissolved in 0.1 M PBS at a concentration of 10 mg/mL, and 47.8 μL of 2-mercaptoethanol (2-ME) at 2 mg/mL was added, followed by stirring for 1 hour to prepare a thiol-polypropylene oxide 1.7 k-containing block polymer.

To remove excipients in the adalimumab formulation, dialysis was performed for one day using a dialysis cassette (MWCO: 20 KDa) and 0.1M PBS solvent. After dialysis, an antibody solution was obtained and the antibody was quantified using the BCA assay method. To 4 mg antibody solution, 59 μL of sulfo-SMCC (sulfo-succinimidyl 4-(N-maleimidomethyl)cyclohexane-1-carboxylate) at a concentration of 1 mg/mL was added, and the reaction mixture was stirred for 1 hour to substitute the amine group of the antibody with a maleimide group. The resulting product was purified using a PD-10 column to remove unreacted sulfo-SMCC, 233 μL of a thiol-polypropylene oxide 1.7 k-containing block polymer was added to 4 mL of the maleimide group-substituted antibody solution and stirred at 4° C. for 18 hours. Then, the final compound was obtained by removing unreacted substances through centrifugation (6500 g, 20 minutes) in an Amicon Ultra (2 mL, MWCO: 100 kDa) tube, and then stored in a refrigerator in a solution state.

### Example 1-2: Production and Purification of adalimumab-maleimide-thiol-polypropylene oxide 3.8 k-Containing Block Polymer Conjugate

In 5 mL of dichloromethane (DCM), 1 g of polypropylene oxide 3.8 k-containing block polymer (Poloxamer 407), 320 mg of 4-nitrophenyl chloroformate (4-NPC), and 1.9 mg of 4-dimethylaminopyridine (DMAP) were dissolved and stirred at 4° C. for 30 minutes, and then stirred again at room temperature for 12 hours to react. After precipitating 5 mL of the reaction solution in 40 mL of diethylether (DE), the reaction product was subjected to centrifugation (3500 rpm, 5 minutes) to remove the supernatant and obtain the precipitate. The resulting product was centrifuged three times and then dried under reduced pressure to remove residual reagents and residual DE. The dried compound (500 mg) was dissolved in 5 ml of DCM, 75.8 μL of ethylenediamine (EDA) was then added, and the resulting mixture was stirred for about 12 hours to react. The reaction solution was dialyzed using a dialysis membrane (MWCO: 3500 Da) for one day to obtain an amine-polypropylene oxide 3.8 k-containing block polymer, freeze-dried, and stored in powder form. The results were confirmed through nuclear magnetic resonance spectrum (¹H-NMR) as shown in FIG. 1(b).

After that, 4 mg of amine-polypropylene oxide 3.8 k-containing block polymer was dissolved in 0.1 M PBS at a concentration of 10 mg/mL, and 42.9 μL of 2-mercaptoethanol (2-ME) at 2 mg/mL was added, followed by stirring for 1 hour to prepare a thiol-polypropylene oxide 3.8 k-containing block polymer.

To remove excipients in the adalimumab formulation, dialysis was performed for one day using a dialysis cassette (MWCO: 20 KDa) and 0.1 M PBS solvent. After dialysis, an antibody solution was obtained and the antibody was quantified using the BCA assay method. To 4 mg antibody solution, 59 μl of sulfo-SMCC (sulfo-succinimidyl 4-(N-maleimidomethyl)cyclohexane-1-carboxylate) at a concentration of 1 mg/mL was added, and the reaction mixture was stirred for 1 hour to substitute the amine group of the antibody with a maleimide group. The resulting product was purified using a PD-10 column to remove unreacted sulfo-SMCC, 347 μL of a thiol-polypropylene oxide 3.8 k-containing block polymer was added to 4 mL of the maleimide group-substituted antibody solution and stirred at 4° C. for 18 hours. Then, the final compound was obtained by removing unreacted substances through centrifugation (6500 g, 20 minutes) in an Amicon Ultra (2 mL, MWCO: 100 kDa) tube, and then stored in a refrigerator in a solution state.

### Example 2: Production and Purification of infliximab-maleimide-thiol-polypropylene Oxide 1.7 k-Containing Block Polymer Conjugate

The infliximab-maleimide-thiol-polypropylene oxide 1.7 k-containing block polymer was produced by using the same production method as for the adalimumab-maleimide-thiol-polypropylene oxide 1.7 k-containing block polymer conjugate (Example 1-1), but using infliximab instead of adalimumab as the antibody type.

### Example 3: Production and Purification of golimumab-maleimide-thiol-polypropylene Oxide 1.7 k-Containing Block Polymer Conjugate

The golimumab-maleimide-thiol-polypropylene oxide 1.7 k-containing block polymer was produced by using the same production method as for the adalimumab-maleimide-thiol-polypropylene oxide 1.7 k-containing block polymer conjugate (Example 1-1), but using golimumab instead of adalimumab as the antibody type.

### <Experimental Example 1> Analysis of Whether Antibody-Linker-Polypropylene Oxide-Containing Block Polymer Conjugates are Synthesized

### Experimental Example 1-1: Molecular Weight Measurement Using MALDI-TOF Mass Spectrometry

In order to confirm whether the antibody-linker-polypropylene oxide-containing block polymer conjugates produced in Examples 1-1, 2, and 3 above were polymer-conjugated, the molecular weight was measured using a MALDI-TOF/MS device.

For MALDI-TOF/MS analysis, each sample was quantified by BCA assay and prepared at a concentration of 1 mg/mL based on the antibody. The MALDI TOF Voyager DE-STR from Applied Biosystems was operated in positive mode and linear mode, utilizing sinapinic acid (SA) as the matrix.

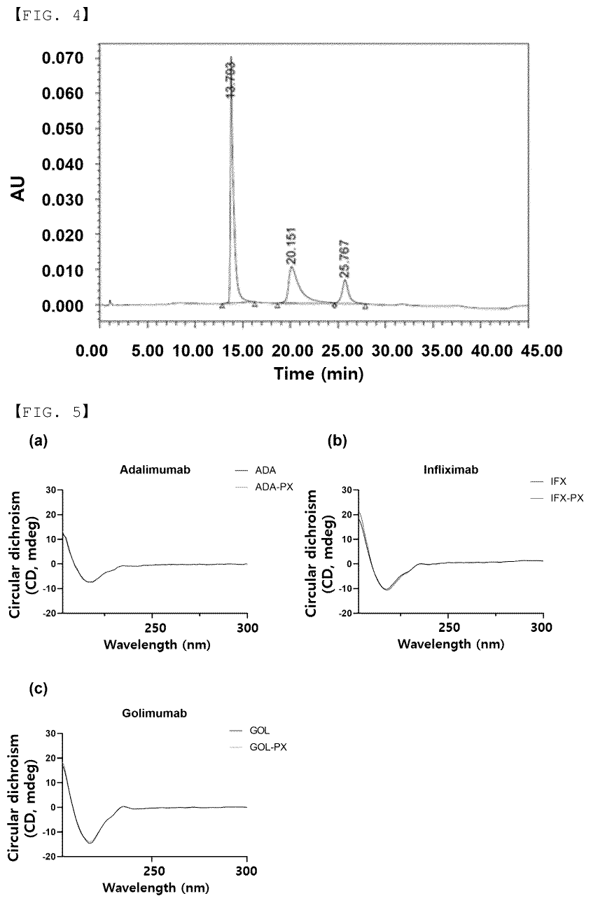

As a result of MALDI-TOF/MS analysis, as shown in FIG. 2, the molecular weights of the adalimumab-polypropylene oxide 1.7 k-containing block polymer, the infliximab-polypropylene oxide 1.7 k-containing block polymer, and the golimumab-polypropylene oxide 1.7 k-containing block polymer were measured to be approximately 157 kDa, 158 kDa, and 159 kDa, respectively. This is similar to the molecular weights of adalimumab (148 kDa) infliximab (149 kDa) and golimumab (150 kDa) plus one molecule of amine-polypropylene oxide 1.7 k-containing block polymer (8640 Da), respectively, confirming the synthesis of antibody-linker-polypropylene oxide-containing block polymer conjugates.

### Experimental Example 1-2: Molecular Weight Measurement Using SDS-PAGE Analysis

In order to confirm whether the antibody-linker-polypropylene oxide-containing block polymer conjugates produced in Examples 1-1, 2, and 3 above were polymer-conjugated, the molecular weight was measured using SDS-PAGE.

For SDS-PAGE analysis, the final concentration of each sample was set to 0.5 mg/mL and 0.25 mg/mL and mixed with loading buffer. For reducing conditions, 5X loading buffer containing 30% glycerol and 0.05% dithiothreitol (DTT) was used. Protein staining was performed using Coomassie blue, while using Mini-PROTEAN TGX gels (4-15% gradient) from BIORAD. Then, the staining was removed and the gel was photographed using a chemiluminescence image analysis device (Chemi Image Documentation System)

SDS-PAGE analysis revealed, as shown in FIG. 3, a band of the antibody heavy chain at about 50 kDa in the sample under reducing conditions, and two bands in the upper part, which were assumed to be antibody-linker-polypropylene oxide-containing block polymer conjugate with increased molecular weight due to conjugation to the polypropylene oxide-containing block polymer. These results confirmed that adalimumab, infliximab, and golimumab were each conjugated with one to three polypropylene oxide-containing block polymer molecules.

### Experimental Example 1-3: Quantification of Conjugates Using Liquid Chromatography

In order to confirm whether the antibody-linker-polypropylene oxide-containing block polymer conjugate produced in Example 1-1 above was polymer-conjugated, the conjugate was quantified employing hydrophobic interaction chromatography (HIC) HPLC columns.

HIC-HPLC analysis was performed using Waters' W2690/5 separation module, and samples were measured at 280 nm using a W2489 UV/Visible detector. The column used was MAbPac HIC-Butyl column 5 μm, 4.6×100 mm from Thermo Fisher Scientific, and the solvents used for the analysis were 1.5 M ammonium sulfate, 50 mM sodium phosphate (pH 7.0) and 50 mM sodium phosphate (pH 7.0): isopropyl alcohol in a ratio of 8:2. The flow rate was set to 0.8 mL/min and the column temperature was maintained at 25° C. during the analysis. The analysis was conducted using 5, 2.5, 1, 0.5, 0.25, 0.1, 0.05, 0.025, 0.01, and 0 mg/mL of the existing antibody as references.

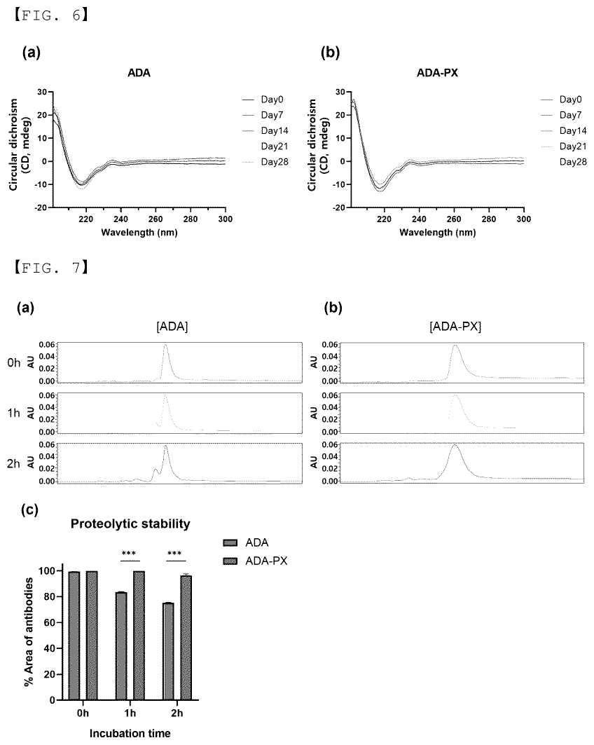

HIC-HPLC analysis showed that the antibody peak for the standard appeared at 13 minutes, as shown in FIG. 4. The antibody-linker-polypropylene oxide-containing block polymer conjugate showed a peak at 13 minutes, similar to the peak of the conventional antibody without polypropylene oxide-containing block polymer conjugation, and peaks at 20 minutes and 25 minutes, respectively. Considering the increased hydrophobicity upon conjugation of polypropylene oxide-containing block polymer, each peak was judged to be the antibody-linker-polypropylene oxide-containing block polymer conjugate containing one or two polypropylene oxide-containing block polymers, respectively.

The MALDI-TOF-MS, SDS-PAGE, and HIC-HPLC analysis results confirmed the same conjugation trend, indicating that the antibody-linker-polypropylene oxide-containing block polymer conjugate was synthesized.

### <Experimental Example 2> Stability Evaluation of Antibody-Linker-Polypropylene Oxide-Containing Block Polymer Conjugates

### Experimental Example 2-1. Stability Evaluation Using Circular Dichroism Spectrometry

Circular dichroism spectrometer (CD) analysis was performed to determine whether the secondary structures of the antibody-linker-polypropylene oxide-containing block polymer conjugates produced in Examples 1-1, 2, and 3 above were modified.

The antibody and antibody-linker-polypropylene oxide-containing block polymer conjugates were quantified by BCA assay and prepared at a concentration of 0.5 mg/mL based on the antibody, and the CD spectra were analyzed. The analysis was performed in the 200 nm-300 nm range, with a total of 10 accumulations.

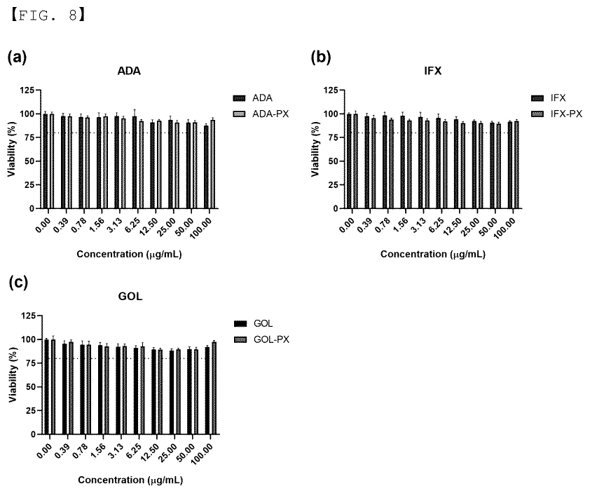

The CD spectra of the antibody and the antibody-linker-polypropylene oxide-containing block polymer conjugate were checked, and both showed the beta-sheet structure and disulfide bond peaks that specifically appear in the antibodies as shown in FIG. 5, indicating that there was no modification of the secondary structure through the fact that there was no difference in the spectra of the antibody and the antibody-linker-polypropylene oxide-containing block polymer. Therefore, it was determined that the antibody-linker-polypropylene oxide-containing block polymers were capable of interacting with the antigens.

### Experimental Example 2-2. Evaluation of Stability in Biological Condition

In order to evaluate the stability of the antibody-linker-polypropylene oxide-containing block polymer conjugate produced in Example 1-1 above compared to the conventional antibody, the antibody-linker-polypropylene oxide-containing block polymer conjugate and the conventional antibody in PBS were compared in view of the structural stability.

The antibody and antibody-linker-polypropylene oxide-containing block polymer were prepared at a final concentration of 0.5 mg/ml and cultured at 37° C. for 4 weeks. The stability of the antibody was confirmed by examining the change in secondary structure over time through the circular dichroism spectrometry used in Experimental Example 2-1 above.

Evaluation of antibody stability in PBS showed that both the conventional antibody and the antibody-linker-polypropylene oxide-containing block polymer conjugate showed no significant change in CD spectra compared to day 0, as shown in FIG. 6. This suggests that the antibody-linker-polypropylene oxide-containing block polymer conjugates can be expected to retain the secondary structure of the protein in vivo for 4 weeks or more, similar to conventional antibodies.

### Experimental Example 2-3. Evaluation of Degradation Stability in Proteolytic Enzyme

In order to evaluate the stability of the antibody-linker-polypropylene oxide-containing block polymer conjugate produced in Example 1-1 above compared to the conventional antibody, the conventional antibody and the antibody-linker-polypropylene oxide-containing block polymer conjugate were treated with a proteolytic enzyme. Proteinase K was selected as the proteolytic enzyme.

Proteinase K was prepared at 250 μg/ml in 1×HBSS, 1 mM EDTA, and the antibody was prepared at 4 mg/ml using conventional buffer, 0.1 M PBS. The two solutions were mixed in a 1:1 volume ratio and treated at 37° C. for 0, 1, and 2 hours, respectively. Then, 4-benzenesulfonyl fluoride (AEBSF) was added to the final solution to 100 mM to terminate the action of the proteolytic enzyme. Each sample was mixed with an equal volume of mobile phase solvent for analysis by reverse phase high performance liquid chromatography (RP-HPLC). Measurements were conducted using a concentration gradient where the column used was a Thermo MAbPac RP column (2.1 mm, 100 mm), and the mobile phase solvents were water (0.1% formic acid) and acetonitrile (0.1% formic acid), respectively. The instrumental analysis was performed at 65° C. at a flow rate of 0.4 ml/min.

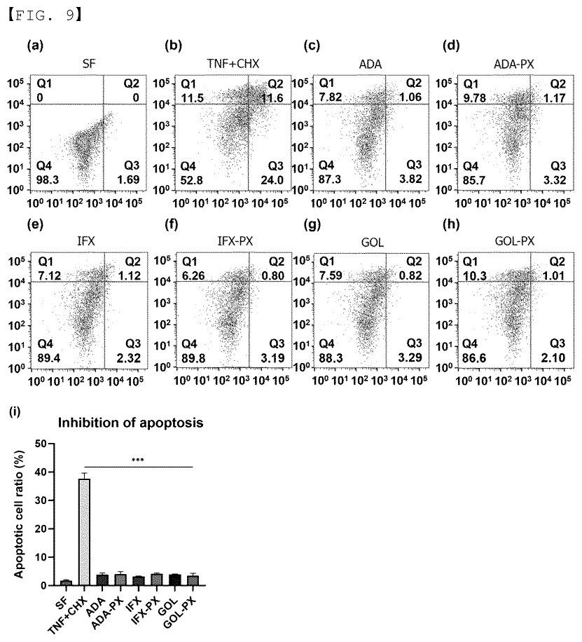

Evaluation of enzymatic protein degradation stability showed that the degree of degradation of proteinase K over time was reduced in the antibody-linker-polypropylene oxide-containing block polymer conjugate compared to the conventional antibody, as shown in FIG. 7. It was determined that the antibody degradation action was inhibited by the ‘steric hindrance’ effect, which physically hinders the access of the degradation enzyme by conjugating the polypropylene oxide-containing block polymer to the antibody. This suggests that the antibody-linker-polypropylene oxide-containing block polymer conjugates are expected to be more stable in vivo than conventional antibodies.

### <Experimental Example 3> Determination of Cytotoxicity and In Vivo TNF-α Neutralizing Effect of Antibody-Linker-Polypropylene Oxide-Containing Block Polymer Conjugates

Cell experiments were performed to confirm that the antibody-linker-polypropylene oxide-containing block polymer conjugates produced in Examples 1-1, 2, and 3 retained the ability to neutralize TNF-α and exhibited no toxicity.

To determine the cytotoxicity of the antibody-linker-polypropylene oxide-containing block polymer conjugates, the toxicity was evaluated on the mouse fibroblast cell line L929. L929 was cultured in RPMI medium containing 10% FBS and 1% penicillin-streptomycin. The experiment was conducted by seeding cells at a density of 1×10⁴ cells in a 96-well plate. Then, each cell group was treated with a concentration of up to 100 mg/ml on a protein basis. After incubation at 37° C. and 5% CO₂ for 24 hours, the culture medium was removed, washed once with Dulbecco's phosphate-buffered saline (DPBS), and subjected to MTT assay. After treatment with MTT reagent (0.4 mg/mL), the cells were cultured at 37° C. in 5% CO₂ for 4 hours. The reagent was removed, formazan was dissolved in DMSO, and cell viability was analyzed by measuring absorbance at 570 nm using a microplate reader. As shown in FIG. 8, the toxic effects on normal cells were first determined and then compared to the cytotoxicity of conventional antibodies and antibody-linker-polypropylene oxide-containing block polymers. The results showed that all the conventional antibodies and the antibody-linker-polypropylene oxide-containing block polymers did not exhibit significant cytotoxicity levels, even up to 100 μg/ml.

To confirm that the biological function of the antibody-linker-polypropylene oxide-containing block polymer conjugates were maintained, the TNF-α neutralizing effect was then tested in vivo. The cell line was the HaCaT cell line, a human keratinocyte, cultured in a medium containing 10% FBS and 1% penicillin-streptomycin in DMEM. The experiment was conducted by seeding cells at a density of 5×10⁴ cells in a 6-well plate. Recombinant human TNF-α and cycloheximide (CHX) were used to induce cell apoptosis.

The experiment was conducted by dividing the cells into four groups: a negative control group (SF) that was not treated with TNF-α and CHX, a positive control group treated with both TNF-α and CHX (TNF+CHX), a positive control group treated with conventional antibodies (ADA, IFX, GOL), and a positive control group treated with the antibody-linker-polypropylene oxide-containing block polymer conjugate (ADA-PX, IFX-PX, GOL-PX). Each antibody and antibody-linker-polypropylene oxide-containing block polymer conjugate was quantified using a BCA assay and treated to each cell group at a concentration of 1 μg/ml on a protein basis. TNF-α was treated at 50 ng/ml, and CHX was treated at 5 ξg/mL. The cells were cultured for 24 hours at 37° C. and 5% CO₂, and subjected to AnnexinV-PI staining to confirm the degree of cell apoptosis. The cells in each group that were detached with trypsin-EDTA treatment from the culture medium were centrifuged (2000 rpm, 5 minutes), and washed with DPBS (5% FBS). Then, each group was treated with AnnexinV-FITC and

PI reagents according to the instructions for the AnnexinV-PI staining kit (Abcam, ab14085), and subjected to flow cytometry analysis using a flow cytometer (Becton Dickinson Sciences, FACS Canto II).

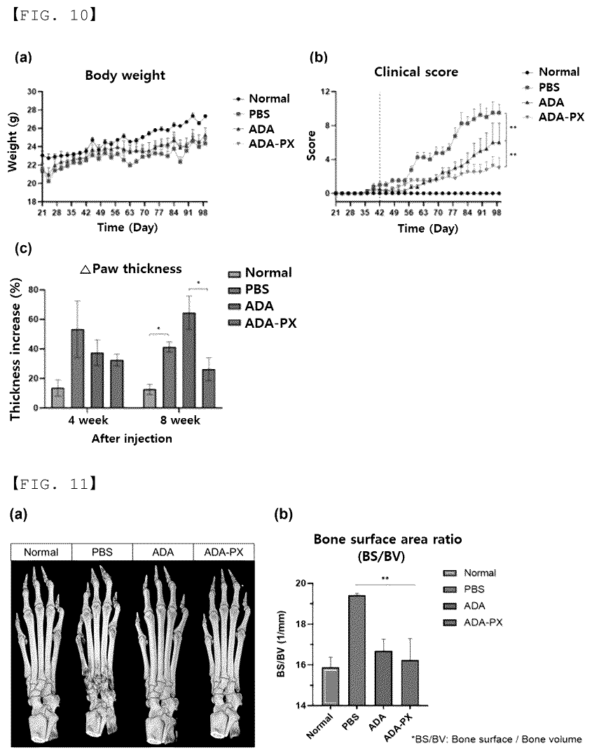

As a result, as shown in FIG. 9, the positive control group (TNF+CHX) showed the increased fluorescence of AnnexinV and PI, confirming that apoptosis was well induced by TNF-α and CHX. Next, the groups treated with conventional antibodies (ADA, IFX, GOL) and the groups treated with antibody-linker-polypropylene oxide-containing block polymer conjugates (ADA-PX, IFX-PX, GOL-PX) showed a decrease in apoptosis compared to the positive control group (TNF+CHX). This is because the conventional antibody inhibited apoptosis by neutralizing recombinant TNF-α, and the absence of significant differences between the conventional antibody-treated groups (ADA, IFX, GOL) and the antibody-linker-polypropylene oxide-containing block polymer conjugate-treated groups (ADA-PX, IFX-PX, GOL-PX) confirmed that the antibody-linker-polypropylene oxide-containing block polymer conjugates exhibited a similar level of TNF-α neutralizing effect as the conventional antibodies.

### <Experimental Example 4> Anti-Inflammatory Effect of Antibody-Linker-Polypropylene Oxide-Containing Block Polymer Conjugates

### Experimental Example 4-1. Evaluation of Anti-Inflammatory Effects in Collagen-Induced Arthritis (CIA) Model

To compare the anti-inflammatory effects of conventional antibodies and antibody-linker-polypropylene oxide-containing block polymer conjugates, collagen-induced arthritis (CIA) DBA/1J mouse mice were created. On day 0, the CIA model was created by administering 100 μl of an emulsion containing collagen type-II and complete Freund's adjuvant mixed in a 1:1 volume ratio to the tail. On day 21, 100 μl of an emulsion containing collagen type-II and incomplete Freund's adjuvant mixed in a 1:1 volume ratio was administered to the tail to induce inflammation to accelerate the progression of arthritis pathology. The sample was administered by intraperitoneal injection at a dose of 10 mg/kg at the time point when inflammation was determined to be induced on average in the model. The thickness of the paw and ankle and the edema formed were checked every 3 days to determine disease progression after inflammation induction, and body weight was measured to determine inflammation-induced weight loss. The thickness of the paw and ankle were measured at the same location with a vernier caliper. Arthritis levels were scored based on the number of edemas formed on the toes and ankle edema. An individual with no edema was given a score of 0; 1 point was assigned if the edema occurred in 1-2 toes, 2 points if the edema occurred in 3-4 toes, 3 points if the edema occurred in all toes, and 4 points if the edema occurred in all toes along with joint stiffness in the ankle and paw. Then, leg tissues were collected, followed by H&E staining to determine the level of inflammation, Safranin O staining to determine cartilage injury, and micro-CT imaging to determine the level of bone erosion.

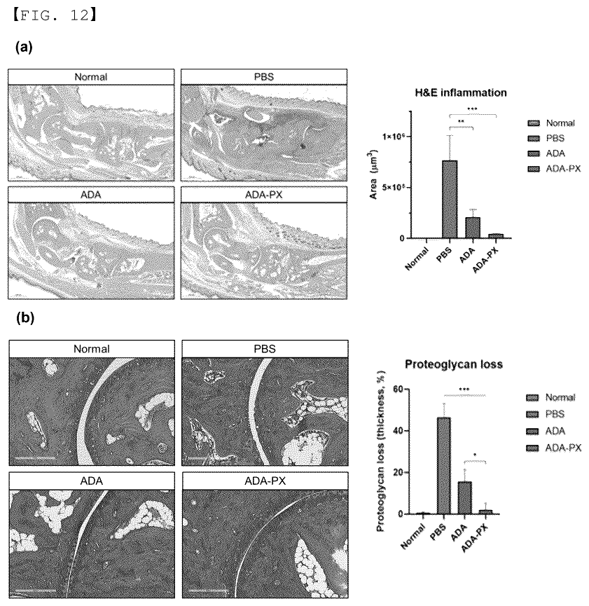

As shown in FIG. 10, the treatment effect on the CIA model showed a significant reduction in arthritis levels in the mice treated with the antibody-linker-polypropylene oxide-containing polymer conjugate compared to the control group, with a difference in effectiveness between the conventional antibody and the antibody-linker-polypropylene oxide-containing polymer conjugate over time. In the case of ankle thickness, a significant reduction was also seen in the mice treated with the antibody-linker-polypropylene oxide-containing block polymer conjugate compared to the control group, and the effectiveness of the antibody-linker-polypropylene oxide-containing block polymer conjugate compared to the conventional antibody was significantly higher at week 8 than at week 4. This was attributed to the increased half-life of the antibody-linker-polypropylene oxide-containing block polymer conjugate in the body compared to conventional antibodies, resulting in improved therapeutic efficacy. Body weight was highest in the normal group and lowest in the control group throughout the entire period, and was similar in the conventional antibody-treated and antibody-linker-polypropylene oxide-containing block polymer conjugate-treated groups.

Anatomical analysis of the CIA model showed that as shown in FIG. 11, the surface area/volume of bone, which indicates the level of bone erosion, was lower in the antibody-linker-polypropylene oxide-treated group compared to the arthritis-induced group, with values similar to the normal group.

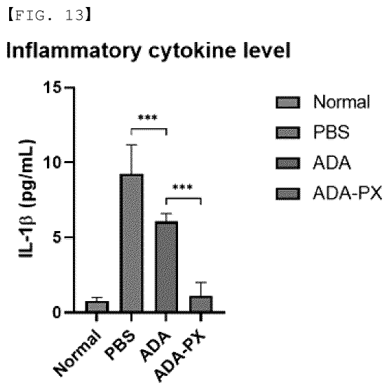

Histological analysis of the CIA model showed that as shown in FIG. 12, the group treated with the antibody-linker-polypropylene oxide-containing block polymer conjugate had significantly lower levels of inflammation, and cartilage and bone injury compared to the antibody-treated group and the control group.

### Experimental Example 4-2. Determination of Inflammatory Factor Expression Levels Through ELISA Analysis

Enzyme-linked immunosorbent assay (ELISA) analysis was performed to confirm the expression levels of inflammatory factors through TNF-α neutralization. For analysis of inflammatory factors, serum was collected from the blood of mice in the last week of the experiment and subjected to ELISA analysis to determine the expression levels of interleukin-1β (IL-1β), an inflammatory cytokine. All ELISA kits were from Abcam and the assays were performed according to the kit instructions.

ELISA analysis confirmed that as shown in FIG. 13, IL-1β expression levels were significantly lower in the adalimumab-linker-polypropylene oxide-containing block polymer conjugate-treated group compared to the negative control group.

In the specification, details capable of being sufficiently recognized and inferred by those skilled in the art of the present disclosure are omitted, and various modifications can be made within the scope that does not change the technical spirit or essential configuration of the present disclosure other than the specific examples described in the present specification. Therefore, the present disclosure may be practiced in other ways than specifically described and exemplified herein, which can be understood by those skilled in the art.

1.-16. (canceled)

17. A conjugate comprising:
(a) an antibody;
(b) a linker linked by a covalent bond to the antibody; and
(c) polyethylene oxide (PEO) and polypropylene oxide (PPO) polymer linked by a covalent bond to the linker.

18. The conjugate of claim 17, wherein the antibody (a) is a TNF-α specific antibody.

19. The conjugate of claim 17, wherein the antibody (a) is selected from the group consisting of Adalimumab, Infliximab, and Golimumab.

20. The conjugate of claim 17, wherein the linker (b) is selected from the group consisting of maleimide-thiol, thiol, maleimide, succinic anhydride, N-hydroxysuccinimide ester, carboxyl-amine, hydrazone, and disulfide bonds.

21. The conjugate of claim 17, wherein the polyethylene oxide (PEO) and polypropylene oxide (PPO) polymer (c) has a weight average molecular weight of 1 kDa to 20 kDa.

22. The conjugate of claim 17, wherein the antibody (a) is selected from the group consisting of Adalimumab, Infliximab, and Golimumab,
wherein the linker (b) is selected from the group consisting of maleimide-thiol, thiol, maleimide, succinic anhydride, N-hydroxysuccinimide ester, carboxyl-amine, hydrazone, and disulfide bonds, and
wherein the polyethylene oxide (PEO) and polypropylene oxide (PPO) polymer (c) has a weight average molecular weight of 1 kDa to 20 kDa.

23. The conjugate of claim 17, wherein the covalent bond is selected from the group consisting of a thioether bond, an amide bond, a carbonyl bond, an ester bond, a thioester bond, a sulfonamide bond, and a urethane bond.

24. The conjugate of claim 21, wherein the polyethylene oxide (PEO) and polypropylene oxide (PPO) polymer is PEO-PPO-PEO polymer.

25. The conjugate of claim 24, wherein the PEO-PPO-PEO polymer is Poloxamer 188 (Pluronic F-68) or Poloxamer 407 (Pluronic F-127).

26. A method for producing a conjugate comprising:
(a) preparing a first conjugate in which a PEO (polyethylene oxide) and PPO (polypropylene oxide) polymer and a linker are linked by a covalent bond; and
(b) preparing a second conjugate in which the linker of the first conjugate and an antibody are linked by a covalent bond.

27. A method for treating an autoimmune disease, comprising: administering a therapeutically effective amount of a conjugate to a subject in need thereof, the conjugate comprising: (a) an antibody; (b) a linker linked by a covalent bond to the antibody; and (c) polyethylene oxide (PEO) and polypropylene oxide (PPO) polymer linked by a covalent bond to the linker.

28. The method of claim 27, wherein the autoimmune disease is at least any one selected from the group consisting of atopic dermatitis, alopecia areata, allergies, asthma, conjunctivitis, periodontitis, rhinitis, otitis media, pharyngitis, tonsillitis, pneumonia, gastric ulcer, gastritis, Crohn's disease, psoriasis, ulcerative colitis, Behcet's enteritis, hidradenitis suppurativa, uveitis, hemorrhoids, gout, ankylosing spondylitis, rheumatic fever, lupus, fibromyalgia, psoriatic arthritis, axial spondyloarthritis, osteoarthritis, rheumatoid arthritis, periarthritis, tendonitis, tenosynovitis, myositis, hepatitis, cystitis, nephritis, Sjogren's syndrome, and multiple sclerosis.

29. The conjugate of claim 27, wherein the antibody (a) is a TNF-α specific antibody.

30. The conjugate of claim 27, wherein the antibody (a) is selected from the group consisting of Adalimumab, Infliximab, and Golimumab.

31. The conjugate of claim 27, wherein the linker (b) is selected from the group consisting of maleimide-thiol, thiol, maleimide, succinic anhydride, N-hydroxysuccinimide ester, carboxyl-amine, hydrazone, and disulfide bonds.

32. The conjugate of claim 27, wherein the polyethylene oxide (PEO) and polypropylene oxide (PPO) polymer (c) has a weight average molecular weight of 1 kDa to 20 kDa.

33. The conjugate of claim 27, wherein the antibody (a) is selected from the group consisting of Adalimumab, Infliximab, and Golimumab,
wherein the linker (b) is selected from the group consisting of maleimide-thiol, thiol, maleimide, succinic anhydride, N-hydroxysuccinimide ester, carboxyl-amine, hydrazone, and disulfide bonds, and
wherein the polyethylene oxide (PEO) and polypropylene oxide (PPO) polymer (c) has a weight average molecular weight of 1 kDa to 20 kDa.

34. The conjugate of claim 27, wherein the covalent bond is at least selected from the group consisting of a thioether bond, an amide bond, a carbonyl bond, an ester bond, a thioester bond, a sulfonamide bond, and a urethane bond.

35. The conjugate of claim 27, wherein the the polyethylene oxide (PEO) and polypropylene oxide (PPO) polymer is PEO-PPO-PEO polymer.

36. The conjugate of claim 35, wherein the PEO-PPO-PEO polymer is Poloxamer 188 (Pluronic F-68) or Poloxamer 407 (Pluronic F-127).
